(19) United States
(12) Patent Application Publication
(10) Pub. No.: US 2007/0243147 A1
(43) Pub. Date: Oct. 18, 2007

(54) Skin and/or Hair Composition Containing Compounds for Increasing The Tanning of Skin

(75) Inventors: Rainer Wolber, Hamburg (DE); Karen Tom Dieck, Hamburg (DE); Cathrin Scherner, Hamburg (DE); Kathrin Schlenz, Hamburg (DE); Inge Kruse, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/632,188

(22) PCT Filed: Jun. 1, 2005
(86) PCT No.: PCT/EP05/52493

§ 371 Date: Mar. 29, 2007

(30) Foreign Application Priority Data
Jul. 24, 2004 (DE) 10 2004 036 092.8

(51) Int. Cl. A61K 8/00; A61K 31/045

(52) U.S. Cl. 424/59; 514/739

(57) ABSTRACT

The invention relates to agents that are to be applied to the skin or hair and contain compounds for intensifying tanning of the skin and increasing melanin synthesis in skin or hair. The invention particularly relates to cosmetic or dermatological preparations. Using said preparations results in inducing and intensifying the tanning mechanisms of the skin, intensifying the hair color, and thus also increasing intrinsic protection of the skin or hair.

A composition that is suitable for use on skin or hair. The composition comprises one or more compounds of formulae (I) to (VIII) as set forth in the specification and at least one other component or element.

The present invention relates to compositions for application to the skin and/or the hair, in particular to intensify tanning of the skin and melanin synthesis in the skin or the hair. In particular, the invention covers cosmetic or dermatological preparations containing these compounds. The use of the preparations leads to the induction and intensification of the natural tanning mechanisms of the skin, to the intensification of the color of the hair and thus also to an increase of the intrinsic protection of the skin or hair.

The harmful effect of the ultraviolet part of solar radiation on the skin is generally known. Whereas rays with a wavelength of less than about 290 nm (the so-called UVC range) are absorbed by the ozone layer in the earth's atmosphere, rays in the range between about 290 nm and about 320 nm, the so-called UVB range, cause erythema, simple sunburn or even burns of greater or lesser severity on the skin.

Numerous compounds are known for protecting against UVB radiation; these are usually derivatives of 3-benzylidene camphor, of 4-aminobenzoic acid, of cinnamic acid, of salicylic acid, of benzophenone and also of 2-phenylbenzimidazole.

It is also important to have available filter substances for the range between about 320 nm and about 400 nm, the so-called UVA region, since its rays can also cause damage. It has been found that UVA radiation leads to damage of the elastic and collagen fibers of connective tissue, which results in premature aging of the skin, and is to be regarded as a cause of numerous phototoxic and photoallergic reactions. The harmful effect of UVB radiation can be intensified by UVA radiation.

UVA radiation can also cause skin damage by damaging the keratin or elastin present in the skin. As a result, elasticity and the ability of the skin to store water is reduced, i.e. the skin becomes less supple and tends towards wrinkling. This type of wrinkle formation is called skin aging caused by light. The strikingly high incidence of skin cancer in regions where solar radiation is strong indicates that damage to the genetic information in the cells is also obviously caused by sunlight.

However, UV radiation can also lead to photochemical reactions, in which case the photochemical reaction products intervene in the skin's metabolism.

Such photochemical reaction products are predominantly free-radical compounds, for example, hydroxyl radicals. Undefined free-radical photo-products which are formed in the skin itself can also display uncontrolled secondary reactions because of their high reactivity. However, singlet oxygen, a non-radical excited state of the oxygen molecule, can also be formed during UV irradiation, as can short-lived epoxides and many others. Singlet oxygen, for example, differs from the normally present triplet oxygen (free-radical ground state) by its increased reactivity. However, excited, reactive (free-radical) triplet states of the oxygen molecule also exist. Through oxidative damage to various skin structures, processes of this type are an important factor in skin aging (including wrinkling) caused by the sun.

UV radiation is also a type of ionizing radiation. There is therefore the risk that UV exposure may also produce ionic species, which then, for their part, are capable of oxidative intervention in the biochemical processes.

The pigmentation of human skin is essentially brought about by the presence of melanin. Melanin and its degradation products (melanoids), carotene, the degree of perfusion, and the condition and thickness of the stratum corneum and other skin layers permit skin shades from virtually white (in cases of reduced filling or in cases of an absence of blood vessels) or yellowish via pale brown-reddish, bluish to brown of different shades and finally almost black. The individual regions of the skin display differing depths of shade as a result of varying amounts of melanin.

Natural melanin protects the skin from penetrating UV radiation. The number of melanin granules produced in the melanocytes determines whether a person has pale skin or dark skin. In cases of strong pigmentation (e.g., in colored races, but also in those with pale skin following UV irradiation), melanin is also to be found in the stratum spinosum and even in the stratum corneum. It attenuates the UV radiation by up to about 90% before it reaches the corium.

Melanocytes contain, as characteristic cell organelles, melanosomes, in which the melanin is formed. On excitation by UV radiation, among other factors, the formation of melanin is increased. It is transported via the living layers of the epidermis (keratinocytes) ultimately to the horny layer (corneocytes) and induces a more or less pronounced brownish to brown-black skin color. Melanin is formed as the final stage in an oxidative process in which tyrosine, with the assistance of the enzyme tyrosinase, converts via a number of intermediate stages to the brown to brown-black eumelanins (DHICA and DHI melanin) or, with the participation of sulfur-containing compounds, to the reddish pheomelanin. DHICA and DHI melanin are formed via the common intermediate stages of dopaquinone and dopachrome. The latter is converted, partially with participation of further enzymes, either into indole-5,6-quinone-carboxylic acid or into indole-5,6-quinone, from which the two aforementioned eumelanins are formed. Pheomelanin is formed, inter alia, via the intermediates dopaquinone and cysteinyldopa.

Besides various functions of the melanin endogenous to the skin, including “detoxification”/binding of toxic substances/pharmaceuticals, etc., the function of melanin as a natural UV filter to protect against harmful UV rays, and also the antioxidant function of melanin as a protection against reactive oxygen species (oxidative stress), which may arise as a result of solar radiation, among other factors, is very important for the skin, with regard, among other things, to homeostasis, prevention of skin aging, prevention of sunburn, and so on. Hence there should be not only a cosmetic benefit in the sense of enhanced tanning as a result of the increased synthesis of melanin in the skin following topical application of compounds which increase melanogenesis, but also an additional protection as a result of the various protective functions of melanin and its precursors.

The object of the present invention is therefore to provide a composition, in particular, a cosmetic or dermatological preparation, that intensifies the natural tanning of the skin through increased melanin synthesis and at the same time leads to an increased intrinsic protection of the skin.

Depending on their sensitivity to light, the following skin types are generally distinguished:

Skin type I never tans, always burns.

Skin type II hardly tans, burns easily.

Skin type III tans averagely well.

Skin type IV tans easily and lastingly, almost never burns.

Skin type V dark, often almost black skin, never burns.

The natural shielding against harmful UV radiation is a manifest advantage of natural skin tanning. For a number of decades now, moreover, a “healthy” skin color has been seen as a sign of athletic activity, in particular, and is therefore regarded as desirable by a broad stratum of consumers. Representatives of skin types I and II who wish to enjoy this type of tan are therefore driven in any case to rely on self-tanning products. However, representatives of skin type III who do not want to be exposed excessively to the risks of sunbathing but nevertheless want to appear tanned, are also appreciative target groups for self-tanning preparations.

The easiest way of giving one's skin a tan is to apply appropriately colored make-up products. Naturally, however, the only parts of the body colored are those covered by the colored products. With the aid of make-up products which can be removed by washing it is possible to achieve a slight skin coloring (for example, extracts of fresh green walnut shells, and henna). One disadvantage of make-up is therefore the time-consuming application process. A further disadvantage is that they strongly stain textiles such as shirt collars or blouses. Furthermore, the various dyes may have different allergenic potential and may even have an irritant effect on the skin.

It is therefore also the object of the present invention to provide preparations that do not exhibit the disadvantages of cosmetic tanning preparations.

Artificial skin tanning can be brought about by cosmetic or medicinal means, with the following approaches essentially playing a part:

The regular intake of carotene products results in carotene being stored in the subcutaneous fatty tissue, and the skin gradually turns orange to yellow-brown.

Coloring can also be accomplished by means of a chemical change in the horny layer of the skin using so-called self-tanning preparations. The principal active substance is dihydroxyacetone (DHA). The tan achieved in this way cannot be removed by washing and comes off only with the normal flaking of the skin (after about 10-15 days). Dihydroxyacetone can be referred to as ketotriose and, as a reducing sugar, reacts with the amino acids of the skin and with the free amino and imino groups of keratin via a series of intermediate stages, in a Maillard reaction, to form brown-colored substances, referred to as melanoids, which are occasionally also called melanoidins.

A particular disadvantage of tanning with dihydroxyacetone is that, unlike “sun-tanned” skin, skin tanned with DHA is not protected from sunburn.

A further disadvantage of dihydroxyacetone is that, particularly under the effect of ultraviolet radiation, it gives off formaldehyde, albeit in amounts which are usually small. There was therefore an urgent need to provide ways in which the decomposition of dihydroxyacetone can be effectively countered.

DE 10212865 describes cosmetic or dermatological preparations containing 9-retinal and/or 9-retinal alkanolamine Schiff base. The use of these preparations leads to the induction and intensification of tanning mechanisms of the skin and intensification of hair color.

One object of the present invention is therefore also to find alternatives to the self-tanning agents known from the prior art which in particular do not have negative properties as are known with DHA.

Coloration by means of self-tanning compositions takes place without exposure to sunlight. In contrast to this, so-called pre-tan products or tan promoters are also offered, which have to be applied prior to exposure to the sun. In the sun, a yellowing of these preparations then arises, which is said to lead to a slight brown-yellow coloration of the outer skin, which additionally enhances the “suntan.”

A further type of artificial tanning which is likewise completely independent of UV light can be brought about by the hormones which are usually released within the body also as a result of (natural) UV exposure and ultimately stimulate the melanocytes to synthesize melanin. In this connection, mention may be made, for example, of modifications of proopiomelanocortin (POMC), such as aMSH and synthetic variants (such as NDP), some of which have much higher activity than the natural aMSH. Although tanning can in principle be brought about by these hormones, their use in cosmetics is not possible since they are clearly pharmacologically effective substances (hormones) which should not be used widely without medicinal indication.

The object of the present invention was likewise to eliminate the disadvantages of the prior art.

In cosmetics, in addition to skin health and skin care, hair care is also a field that is subject to particularly intensive research.

Hair is the thread-like skin appendage which is virtually universal (lacking on palms of the hand, soles of the feet, extensor sides of the distal phalanges of the toes and fingers); differentiated as long hair (head hair, beard hair, axilla hair, pubic hair=capilli, barba, hirci and pubes, respectively; in men also chest hair), short, bristle hair (supercilia, cilia, vibrissae, tragi) and down (lanugo, velus hair). The structure of all these hairs is approximately and on the whole similar: in the center the hair medulla (comprising epithelial cells with eosinophilic horny substance granules=trichohyalin granules), surrounded by the hair cortex (comprising keratinized cells; comprises pigments) and the outer skin of the hair (cuticula pili; anuclear epidermis layer) and by layers of the epithelial and connective tissue hair sheath.

The hair is divided into the hair shaft protruding from the skin and the inclined hair root reaching into the subcutis and whose layers correspond approximately to those of the epidermis. The thickened lower root end, the hair bulb, sits on a vascular connective tissue pin, the hair papilla, protruding into it (both as hair base). The bulb in the starting (=anagen) phase of the cyclically repeating hair formation is coated onion-like as a result of the continuous new formation of cells by its near-papillary layer (matrix), then later closed, bulb-like, very keratinized (bulb hair) and is finally, in the end (=telogen) phase, displaced in the direction of the follicle opening by a new hair—starting from a newly forming hair papilla.

Melanin is responsible for personal hair color. The melanin is formed in the melanocytes, cells which arise in the hair bulb associated with the keratinocytes of the hair medulla. Melanocytes contain melanosomes as characteristic cell organelles where the melanin is formed. This is transferred via the long dendrites of the melanocytes to the keratinocytes of the precortical matrix and brings about the more or less marked blond to brown-black hair color. Melanin is formed as the final stage of an oxidative process in which tyrosine converts, with the assistance of the enzyme tyrosinase, via several intermediates to the brown to brown-black eumelanins (DHICA and DHI melanin) and/or, with participation of sulfur-containing compounds, to the reddish pheomelanin. DHICA and DHI melanins arise via the common intermediate stages dopaquinone and dopachrome. The latter is converted, partially with participation of further enzymes, either into indole-5,6-quinonecarboxylic acid or into indole-5,6-quinone, from which the two specified eumelanins form. The formation of pheomelanin proceeds, inter alia, via the intermediate products dopaquinone and cysteinyldopa. Cysteine is additionally necessary when the pheomelanin is to arise for blond and reddish hair.

The eumelanin is the black-brown pigment. It primarily determines the color depth of the hair. In brown and black hair it is present in clearly discernible granules.

Pheomelanin is the red pigment. It is responsible for pale blond, blond and red hair. Due to its structure, this melanin is very much finer and smaller. The various proportions of the melanin types lead to the various hair colors:
Blond hair contains a small amount of eumelanin and a large amount of pheomelanin.
Dark hair contains a large amount of eumelanin and a small amount of pheomelanin.
Red hair likewise has a small amount of eumelanin and a very large amount of pheomelanin.
All shades of hair in between result from varying mixing ratios of the two melanin types.

The pigment formation process can proceed only if sufficient tyrosinase is available. This enzyme is formed more infrequently with increasing age. This then gradually leads to gray hair. The reason: with little tyrosinase, less and less tyrosine is also formed. The production of melanin thus decreases. The lack of melanin is replaced by the inclusion of air bubbles. The hair appears gray.

This process is usually gradual. It starts at the temples and then extends to the entire head of hair. Subsequently, it affects the beard and the eyebrows. In the end, all of the hair on the body is finally gray.

In medical terms, gray hairs are referred to as canities. There are various graying possibilities. Premature graying, from age 20, is also called canities praecox.

Canities symptomatica, or symptomatic graying of the hair, can have various causes. These include:
Pernicious anemia (vitamin B deficiency anemia),
Severe endocrinological disorders, e.g. in the case of thyroid disorders,
Acute febrile illnesses,
Side-effects of pharmaceuticals,
Cosmetics
Metals

Coloring hair, in particular living human hair, using natural dyes, as has been known since antiquity, particularly for the dye henna, and which has been pushed into the background in favor of synthetic dyes, has for some years been the object of new interest. The red shade which arises with henna is a disadvantage.

Melanin production, which produces the hair color, decreases with increasing age: the hair becomes gray or white. It is a cosmetic wish for some consumers to reverse or to slow this process. For this purpose, the cosmetics industry in some countries uses lead acetate which is toxic and is therefore prohibited in the European Cosmetics Directive. This lead acetate is preferably applied in the form of a solution to the hair and remains there for a prolonged period without being washed off.

For dyeing keratin-containing fibers, e.g. hair, wool or furs, use is generally made either of direct dyes or oxidation dyes, which are formed by oxidative coupling of one or more developer components with one another or one or more coupler components. Coupler and developer components are also referred to as oxidation dye precursors.

The developer components used are usually primary aromatic amines with a further free or substituted hydroxyl or amino group, situated in the para or ortho position, diaminopyridine derivatives, heterocyclic hydrazones, 4-aminopyrazolone derivatives, and 2,4,5,6-tetraminopyrimidine and derivatives thereof.

Specific representatives are, for example, p-phenylenediamine, p-tolylenediamine, 2,4,5,6-tetraminopyrimidine, p-aminophenol, N,N-bis(2-hydroxyethyl)-p-phenylenediamine, 2-(2,5-diaminophenyl)ethanol, 2-(2,5-diaminophenoxy)ethanol, 1-phenyl-3-carboxyamido-4-amino-5-pyrazolone, 4-amino-3-methylphenol, 2-aminomethyl-4-aminophenol, 2-hydroxymethyl-4-aminophenol, 2-hydroxy-4,5,6-triaminopyrimidine, 2,4-dihydroxy-5,6-diaminopyrimidine and 2,5,6-triamino-4-hydroxypyrimidine.

Coupler components used are usually m-phenylenediamine derivatives, naphthols, resorcinol and resorcinol derivatives, pyrazolones and m-aminophenols. Suitable coupler substances are, in particular, α-napthol, 1,5-, 2,7-, 1,7-dihydroxynaphthalene, 5-amino-2-methylphenol, m-aminophenol, resorcinol, resorcinol monomethyl ether, m-phenylenediamine, 2,4-diaminophenoxyethanol, 1-phenyl-3-methyl-5-pyrazolone, 2,4-dichloro-3-aminophenol, 1,3-bis(2,4-diaminophenoxy)propane, 2-chlororesorcinol, 4-chlororesorcinol, 2-chloro-6-methyl-3-aminophenol, 2-methylresorcinol and 5-methylresorcinol.

With regard to further customary dye components, reference is made expressly to the series “Dermatology,” published by Ch. Culnan, H. Maibach, Verlag Marcel Dekker Inc., New York, Basel, 1986, Vol. 7, Ch. Zviak, The Science of Hair Care, Ch. 7, pages 248-250 (Direct Dyes), and Ch. 8, pages 264-267 (Oxidation Dyes), and also the “European Inventory of Cosmetic Raw Materials,” 1996, published by the European Commission, obtainable in diskette form from the Bundesverband der deutschen Industrie-und Handelsunternehmen fur Arzneimittel, Reformwaren und Korperpflegemittel e.V., Mannheim.

Although intensive colorations with good fastness properties can be achieved with oxidation dyes, the development of the color generally takes place under the influence of oxidizing agents, such as, for example, H₂O₂, which in some cases can result in damage to the fibers. Furthermore, some oxidation dye precursors or certain mixtures of oxidation dye precursors can occasionally have a sensitizing effect in people with sensitive skin. Although direct dyes are applied under more moderate conditions, their disadvantage is that the colorations frequently have only inadequate fastness properties.

The object of the present invention is to improve hair's independent melanin production, but without having to rely on dyes and in particular oxidants such as, e.g., H₂O₂. Moreover, the agents must not have any or just a very small sensitizing potential.

It is the object of the present invention also to provide alternative agents for tanning the skin or increasing melanin synthesis.

It was now surprisingly found that the entire group of objects is attained with a composition according to claim 1, in particular cosmetic or dermatological preparations according to one of claims 3 through 15.

The subject matter of the subclaims is advantageous embodiments of the compositions according to the invention. Furthermore, the invention covers the use of such compositions for increasing a tan of the skin or the melanin synthesis in the skin or the hair.

It was surprising and not foreseeable for one skilled in the art that the objects are attained with agents for application to the skin and/or the hair, in particular cosmetic or dermatological preparations containing one or more compounds with the structure
(I) (2R,3S,4R)-2,3,4-trimethyl-3-[(3E,7E,11E)-3,8,12,16-tetramethylheptadeca-3,7,11,15-tetraen-1-yl]cylcohexanone with the structure 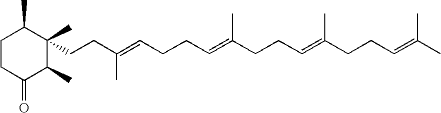
(II) (2E,6E)-10-hydroxy-2,6,10-trimethyldodeca-2,6,11-trien-1-yl acetate with the structure 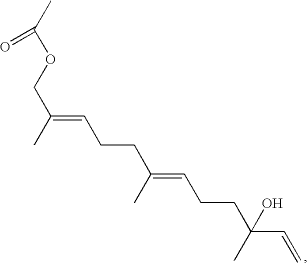
(III) (4E,8E,12E, 16E)-3,7,11,15-tetrahydroxy-18-(hydroxymethyl)-2,4,6,10,14,16,20-heptamethyldocosa-4,8,12,16-tetraenoic acid with the structure 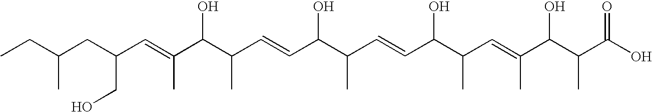
(IV) (3E)-7-hydroxy-1-[(1Z)-3-hydroxy-2-methylprop-1-en-1-yl]-3,7-dimethylnona-3,8-dien-1-yl acetate with the structure 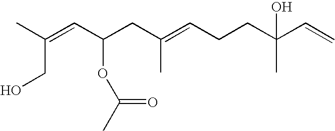
(V) (2E,6Z)-10-hydroxy-2,6,10-trimethyldodeca-2,6,11-trienoic acid with the structure 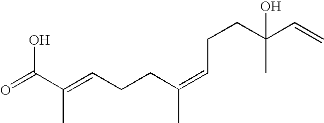
(VI) 6-methyl-8-(2,5,5,8a-tetramethyl-1,4,4a,5,6,7,8,8a-octahydronaphthalen-1-yl)oct-5-en-2-one with the structure 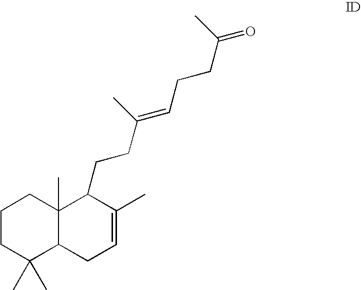
(VII) (2E,6E,10E)-13-[(1R,2R,8aS)-2-hydroxy-2,5,5,8a-tetramethyl-6-oxodeca hydronaphthalen-1-yl]-2,6,10-trimethyltrideca-2,6,10-trienoic acid 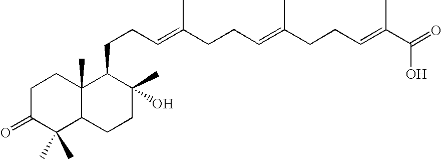
and/or
(VIII) (4aS,5R,6R)-6-hydroxy-5-[(3E,7E,11E)-13-hydroxy-4,8,12-trimethyltrideca-3,7,11-trien-1-yl]-1,1,4a,6-tetramethyloctahydronaphthalen-2(1H)-one

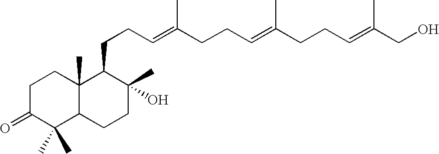

The substances according to the invention and derivatives thereof, which are characterized by the compound structures (I) through (VIII), are extremely suitable for effecting an increased tanning of the skin. All compounds of the previously listed structures which one skilled in the art can chose from the respective groups and combine are shown to be suitable.

In tests to increase the synthesis of melanin, in particular the compounds with the structure (III) (4E,8E,12E,16E-3,7,11,15-tetrahydroxy-18-(hydroxymethyl)-2,4,6,10,14,16,20-heptamethyldocosa-4,8,12,16-tetraenoic acid with the structure 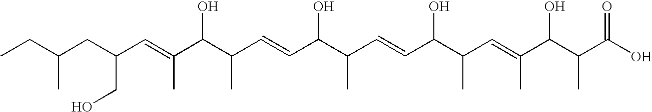
and derivatives thereof have proven to be particularly advantageous.

The melanin endogenous to the skin has various functions, including, for example, “detoxification”/binding of toxic substances/pharmaceuticals. In addition, the function of melanin as a natural UV filter is to protect against harmful UV rays, and also the antioxidant function of melanin as a protection against reactive oxygen species (oxidative stress), which may arise as a result of solar radiation, among other factors, is very important for the skin, with regard, among other things, to homeostasis, prevention of skin aging, prevention of sunburn, etc. There should therefore be not only a cosmetic benefit in the sense of enhanced tanning as a result of the increased synthesis of melanin in the skin following topical application of compounds (I) to (VIII) which increase melanogenesis according to the invention, but also an additional protection as a result of the various protective functions of melanin.

The compounds according to the invention are suitable for intensifying the physiological tanning of the skin via an increased synthesis of melanin and thus also for increasing the intrinsic protection of the skin. A crucial advantage is that this physiological tanning is achieved without having to expose oneself to natural solar radiation with its harmful effects on the skin or that this is necessary only to a comparatively small extent in order to achieve the desired tan. In addition to an increasing tan, uneven skin tone is also corrected. The advantage: the skin appears to be more even, which is desirable in particular with older skin (age spots), melasma and post-inflammatory hyperpigmentation.

The topical application of the compounds according to the invention is in principle possible and preferred in different, in particular W/O as well as O/W formulas and other cosmetic forms of administration.

The subject matter of the invention is therefore preferably cosmetic or dermatological preparations containing compounds according to the invention, as defined above. However, in addition, the compositions can be used in polymer matrices, in a skin patch or wound dressing, a plaster, a wipe or a pad, a spray or in a textile.

The subject matter of the invention is also the use of the application forms and preparations thus produced.

The following compounds have proven to be useful as combination partners, i.e., in addition to compounds (I) and (VIII) in the products according to the invention, which compounds in combination with the structural compounds (I) to (VIII) show a synergistic effect, both with respect to the tanning effect and with respect to the endogenous protection.

From the group of terpenoids, phytoene, 7,7′,8,8′,11,11′,12,12′-octahydro-ψ,ψ-carotene of the structure 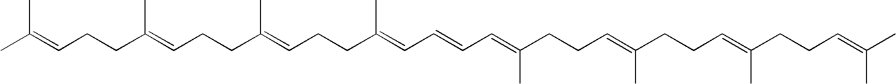
is preferred as combination partner to the compounds according to the invention.

Likewise preferred is phytofluene, 7,7′,8,8′,11,12-hexahydro-ψ,ψ-carotene, of the structure

A further preferred compound is 4-carotene, 7,7′,8,8′-tetrahydro-ψ,ψ-carotene, of the structure

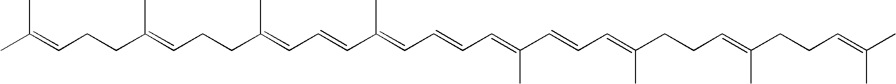

As further combination compounds, compounds are preferably used that can be chosen from the group
neurosporine, 7,8-dihydro-ψ,ψ-carotene, of the structure 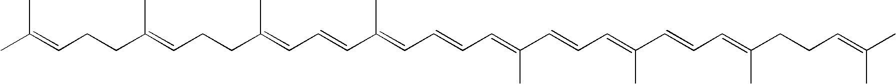
lycopene, ψ,ψ-carotene, 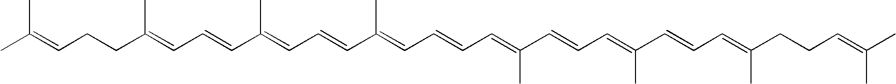
β-carotene, β,β-carotene of the structure 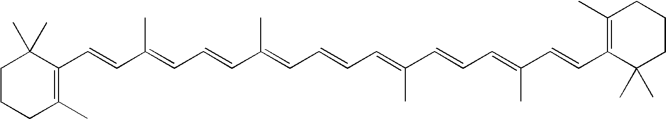
squalene, (6E,10E,14E,18E)-2,6,10,15,19,23-hexamethyltetracosa-2,6,10,14,18,22-hexaene, 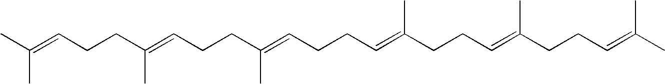
variabilin, (5Z)-5-[(6E,10E)-13-(3-furyl)-2,6,10-trimethyltrideca-6,10-dien-1-ylidene]-4-hydroxy-3-methylfuran-2(5H)-one, 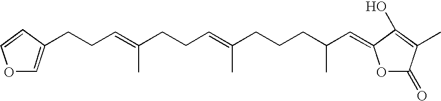
phytanic acid, (3R,7R,11R)-3,7,11,15-tetramethylhexadecanoic acid 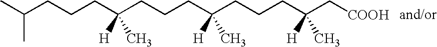
phytol, (2E,7R,11R)-3,7,11,15-tetramethylhexadec-2-en-1-ol, of the structure

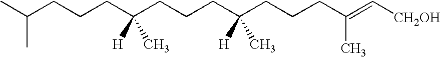

To verify the effectiveness of the compounds (I) to (VIII) according to the invention, effectiveness tests were carried out.

A melanogenesis assay was carried out after 3 days of incubation of primary normal human melanocytes with test substance compared to control. The figures listed in the table give the melanogenesis rates (measured as ¹⁴C-tyrosine incorporation) based on the untreated control (=100%). It results from this that the melanogenesis, i.e., the process of melanine synthesis, rises to 156% or 150% when the melanocytes are cultivated in the presence of the compound (I) (2E,3S,4R)-2,3,4-trimethyl-3-[(3E,7E,11E)-3,8,12,16-tetramethylheptadeca-3,7,11,15-tetraen-1-yl]cyclohexanone (n=4).

| | Control | 1 μg/ml | 0.1 μg/ml |
|---|---|---|---|
| X transverse | 100 | 156 | 150 |
| SEM | 0 | 41 | 45 |

Furthermore, a melanogenesis assay was carried out after 3 days of incubation of primary normal human melanocytes with test substance compared to control. The figures listed in the table give the melanogenesis rates (measured as ¹⁴C-tyrosine incorporation) based on the untreated control (=100%). It results from this that the melanogenesis, i.e., the process of melanine synthesis, rises to 135% or 113% when the melanocytes are cultivated in the presence of the preferred compound (III) (4E,8E,12E,16E)-3,7,11,15-tetrahydroxy-18-(hydroxymethyl)-2,4,6,10,14,16,20-heptamethyldocosa-4,8,12,16-teraenoic acid (n=4).

| | Control | 1 μg/ml | 0.1 μg/ml |
|---|---|---|---|
| X transverse | 100 | 172 | 153 |
| SEM | 0 | 39 | 60 |

These compounds according to the invention have proven useful as agents for application to the skin or the hair. The compounds lead to an increase in the synthesis of melanin and are to be preferably used as sole additives or as a mixture in cosmetic or dermatological preparations.

In addition to the use of the agents as cosmetic or dermatological preparations, a polymer matrix, a skin patch or wound dressing, a plaster, a wipe or a pad, a spray, a stick or textiles, e.g., bandages or bath textiles, is favored as an agent according to the invention in order to ensure continuous tanning. In the case of bandages equipped with the compounds according to the invention, it is advantageous that while the bandage is worn the skin underneath is given a brown coloring just like the uncovered skin.

Intensive research has shown that the compounds according to the invention in compositions to be applied topically, in particular cosmetic or dermatological preparations, lead to the induction of the pigmentation of the skin. The melanogenesis is increased, more melanin is produced in the skin, the skin thus becomes browner and the intrinsic protection of the skin is physiologically increased. In the case of the topical application to the hair, the compounds according to the invention in suitable preparations also lead to an intensification of the hair color, through which a natural graying of the hair can be avoided and even reversed.

Naturally, the activation of the endogenous tanning and the intensification of the hair color can thereby occur with and without the involvement of UV light.

The compounds according to the invention are characterized, inter alia, by the fact that, e.g., following topical application, in the skin they induce the formation of pigments intrinsic to the skin, increase the synthesis of melanin and in this way produce an enhanced tanning of the skin. They are acceptable in terms of health, non-irritative and easy to handle, and the resulting color shade naturally corresponds to that of the natural healthy skin color. The resulting tan, since it corresponds to the natural tan, is lightfast and cannot be washed off. Surprisingly, the agents according to the invention also enhance the tanning of skin which is already tanned and, moreover, delay tanned skin from becoming pale.

A further advantage of the present invention arises from the protective properties of natural melanin formed in the skin. As well as various other functions of the melanin intrinsic to the skin (such as, for example, “detoxification” or binding of toxic substances and/or pharmaceuticals etc.), these functions of melanin are also in particular very important for the skin, inter alia with regard to homeostasis, the prevention of skin aging and the like: melanin acts as a natural UV filter for protection against harmful UV rays, and moreover as an antioxidant for protecting against reactive oxygen species (oxidative stress), which can arise, inter alia, as a result of solar irradiation.

Thus, the use according to the invention, e.g., following topical application results not only in a cosmetic benefit in the sense of enhanced tanning as a result of the increased melanin synthesis in the skin, but also an additional benefit as a result of the various protective powers of melanin.

The compositions according to the invention, cosmetic or dermatological preparations, induce in the skin and in the hair the formation of pigments intrinsic to the skin and the hair, intensify the existing natural and/or artificial tanning of the skin, even out uneven pigmentation of the skin, intensify the natural hair color and prolong the skin tan as well as the hair color.

The formulations according to the invention are entirely satisfactory preparations in every respect which are characterized by a uniformly coloring action. The person skilled in the art could not have foreseen that the formulations according to the invention
Are easier to formulate,
More rapidly and better impart a naturally tanned appearance to the skin and the hair
Prolong skin tan and hair color
Have a better effect than moisturizing preparations,
Better promote skin smoothing, Promote regeneration better
Are characterized by better care action,
Have better sensory properties, such as, for example, ease of distribution on the skin or the ability to be absorbed into the skin, and
Would offer a better/risk-free intrinsic protection of the skin (against UV radiation)
   than the preparations of the prior art. In addition, the formulations according to the invention, surprisingly, do not display any hormone effects.

The content of the compounds (I) to (VIII) is between 0.0001 and 30% by weight, advantageously between 0.001 and 10% by weight, particularly preferably between 0.02 and 2% by weight, respectively based on the total weight of the compositions, preferably of the cosmetic preparations.

As cosmetic and/or dermatological formulation according to the invention they can have the customary composition and be used, in particular, for the treatment and care of the skin and/or the hair, as a make-up product in decorative cosmetics or as a sunscreen preparation or so-called pre-sun or after-sun preparation. Accordingly, the formulations according to the invention can, depending on their formulation, be used, for example, as skin protection cream, face cream, cleansing milk, sunscreen lotion, nutrient cream, or day or night cream, etc.

It is also possible and advantageous for the purposes of the present invention to include the compounds according to the invention in aqueous systems or surfactant preparations for the cleansing and care of the skin and hair. This includes both shower gels, shampoos but also conditioners, hair treatments, hair rinses, hair tonics, sprays etc.

One skilled in the art is, of course, aware that high-quality cosmetic compositions are in most cases inconceivable without the use of the usual auxiliaries and additives. These include, for example, builders, fillers, perfume, dyes, emulsifiers, additional active ingredients such as vitamins or proteins, light protection agents, stabilizers, insect repellents, alcohol, water, salts, substances having anti-microbial, proteolytic or keratolytic activity, preservatives, bactericides, substances for preventing foaming, pigments having a coloring effect, thickeners, humectant and/or moisturizing substances, fats, oils, waxes or other conventional constituents of a cosmetic or dermatological formulation, such a alcohols, polyols, polymers, foam stabilizers, electrolytes, organic solvents, silicone derivatives or moisturizers, etc.

It is also advantageous to provide the compound(s) according to the invention in encapsulated form, for example, in collagen matrices and other common encapsulating materials, for example cyclic oligosaccharides (in particular alpha-, beta-, HP-beta-, random-Me-beta, gamma-cylcodextrin), whereby according to the chemical properties of the compounds according to the invention known to one skilled in the art, alpha-, beta- or gamma-cyclodextrins are used as encapsulating materials. Furthermore, it can be advantageous to provide the compounds according to the invention or mixtures thereof in the form of cellulose encapsulations, in gelatin, wax matrices or liposomally encapsulated.

With encapsulation with cyclodextrins it is assumed that the cyclodextrin structure acts as the host molecule, and the active substance according to the invention, as guest molecule. For production, cyclodextrins are dissolved in water and active substance according to the invention is added. The molecular adduct thereupon precipitates as a solid and can be subjected to customary purification and work-up steps. It is known that cyclodextrin guest complexes in a corresponding solvent (e.g., water) are in an equilibrium between the concrete guest cyclodextrin complex and the dissociated form, whereby cyclodextrin and guest may be separated to a certain extent. Such equilibrium systems are likewise advantageous for the purposes of the present invention.

Corresponding requirements apply mutatis mutandis for the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g., Cosmetics Directive, Foods and Drugs Act).

It is thereby likewise advantageous to add the compound(s) according to the invention as additive to preparations that already contain other active substances for other purposes.

It was thus surprisingly found with the present invention that the formulations according to the invention are particularly well suited for combination with active substances that have a positive effect on the condition of the skin. It was thus shown that active ingredients for positively influencing aging skin which reduce the development of lines or even existing lines. Thus in particular in combination with bioquinones, in particular ubiquinone Q10, creatine, creatinine, carnitine, biotin, isoflavone, cardiolipin, lipoic acid, liponamide, folic acid and its derivatives, niacin and its derivatives (in particular niacinamide), arctiin, antifreezing proteins, hop and hop-malt extracts. Agents which promote the restructuring of connective tissue, such as isoflavonoids and isoflavonoid-containing plant extracts such as, for example, soya and clover extracts can also be used very well in the formulations according to the invention. It is also found that the formulations are particularly suitable for using active ingredients for aiding the skin functions in dry skin such as, for example, vitamin C, biotin, carnitine, creatine, propionic acid, green tea extracts, eucalyptus oil, urea and mineral salts such as, for example, NaCl, sea minerals, and osmolytes such as, for example, taurine, inositol, betaine, quaternary ammonium compounds. In a similar way, the incorporation of active ingredients for alleviating or positively influencing irritative skin conditions, whether for sensitive skin in general or for skin irritated by noxae (UV light, chemicals) has proven to be advantageous. Mention is made here of active ingredients such as sericosides, various extracts of licorice, licochalcones, in particular licochalcone A, silymarin, silyphos, dexpanthenol, inhibitors of prostaglandin metabolism, in particular of cyclooxygenase and of leukotriene metabolism, in particular of 5-lipoxygenase, but also of the 5-lipoxygenase inhibitor protein, FLAP. The incorporation of pigmentation modulators has also proven to be advantageous. Mention is made here of active ingredients which reduce the pigmentation of the skin and thus lead to a cosmetically desired lightening of the skin, reduce the appearance of age spots and/or lighten existing age spots. By way of example, mention may be made of tyrosine sulfate, dioic acid (8-hexadecene-1,16-dicarboxylic acid), and lipoic acid and liponamide, various extracts of licorice, kojic acid, hydroquinone, arbutin, alpha-arbutin, deoxyarbutin, fruit acids, in particular alpha-hydroxy acids (AHAs), bearberry (Uvae ursi), ursolic acid, ascorbic acid, green tea extracts, aminoguanidine, pyridoxamine. In the same way, the formulations according to the invention proved to be excellent combination partners for further active ingredients which bring about an increased or more rapid tanning of the skin, be it with or without the effect of UV light, (Advanced Glycation Endproducts (AGE), lipofuscins, nucleic acid oligonucleotides, duhydroxyacetone, erythrulose, purines and pyrimidines, NO-releasing substances.

Cosmetic and dermatological preparations that are present in the form of a sun screen are particularly preferred. Advantageously, these can additionally contain at least one further UVA filter and/or at least one other UVB filter and/or at least one inorganic pigment, preferably an inorganic micropigment.

Surprisingly, cosmetic and dermatological preparations according to the invention are able to prolong the natural tan.

It is also surprising that cosmetic and dermatological formulations according to the invention are able to help to treat hypopigmentations (vitiligo, uneven pigmentation in aged skin, etc.).

Moisturizers is the term used to describe substances or mixtures of substances which, following application or distribution on the surface of the skin, impart to cosmetic or dermatological preparations the property of reducing the moisture loss by the horny layer (also called transepidermal water loss (TEWL)) and/or positively influencing hydration of the horny layer.

Advantageous moisturizers for the purposes of the present invention are, for example, glycerol, lactic acid, pyrrolidonecarboxylic acid and urea. It is also particularly advantageous to use polymeric moisturizers from the group of polysaccharides which are soluble in water and/or swellable in water and/or gelable using water. Particularly advantageous are, for example, hyaluronic acid and/or a fucose-rich polysaccharide which is listed in Chemical Abstracts under the registry number 178463-23-5 and is available, for example, under the name Fucogel®1000 from SOLABIA S.A.

Glycerin can be used as a moisturizer for the purposes of the present invention in the range of 0.05-30% by weight, particularly preferred is 1-10%.

The amounts of cosmetic or dermatological auxiliaries and carriers and perfume to be used in each case can easily be determined by the person skilled in the art by simple trial and error depending on the type of product in question.

An additional content of antioxidants is generally preferred in the preparations according to the invention. According to the invention, favorable antioxidants which can be used are any antioxidants which are suitable or conventional for cosmetic and/or dermatological applications.

It is therefore advantageous to add antioxidants to the preparations according to the invention. The antioxidants are advantageously chosen from the group consisting of amino acids (e.g., glycine, histidine, tyrosine, tryptophan) and derivatives thereof (in particular N-acetyl tyrosin, N-acetyl phenylalanine), imidazoles (e.g., urocanic acid) and derivatives thereof, peptides such as D,L-carnosine, D-carnosine, L-carnosine and derivatives thereof (e.g., anserine), carotenoids, carotenes (e.g. α-carotene, β-carotene, lycopene) and derivatives thereof, chlorogenic acid and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g., buthionine sulfoximines, homocysteine sulfoximine, buthionine sulfones, penta, hexa-, heptathionine sulfoximine) in very low tolerated doses (e.g., pmol to μmol/kg), and also (metal) chelating agents (e.g., α-hydroxy fatty acids, palmitic acid, phytic acid, lactoferrin), α-hydroxy acids (e.g., citric acid, lactic acid, malic acid), humic acid, bile acid, bile extracts, bilirubin, biliverdin, EDTA, EGTA and derivatives thereof, unsaturated fatty acids and derivatives thereof (e.g., γ-linolenic acid, linoleic acid, oleic acid), folic acid and derivatives thereof, ubiquinone and ubiquinol and derivatives thereof, vitamin C and derivatives (e.g., ascorbyl palmitate, Mg ascorbyl phosphate, ascorbyl acetate), tocopherols and derivatives (e.g., vitamin E acetate), vitamin A and derivatives (in particular vitamin A palmitate) and coniferyl benzoate of gum benzoin, rutinic acid and derivatives thereof, α-glycosylrutin, ferulic acid, furfurylideneglucitol, carnosine, butylated hydroxytoluene, butylated hydroxyanisole, nordihydroguaiacic acid, nordihydroguaiaretic acid, trihydroxybutyrophenone, uric acid and derivatives thereof, mannose and derivatives thereof, zinc and derivatives thereof (e.g., ZnO, ZnSO₄), selenium and derivatives thereof (e.g., selenomethionine), stilbenes and derivatives thereof (e.g., stilbene oxide, trans-stilbene oxide) and the derivatives (salts, esters, ethers, sugars, nucleotides, nucleosides, peptides and lipids) of said active ingredients, which are suitable according to the invention.

The amount of aforementioned antioxidants (one or more compounds) in the preparations is preferably 0.001 to 30% by weight, particularly preferably 0.05 to 20% by weight, in particular 1 to 10% by weight, based on the total weight of the compositions, preferably of the preparation. If vitamin E and/or derivatives thereof are the antioxidant or antioxidants, it is advantageous to choose the respective concentrations thereof from the range of from 0.001 to 10% by weight, based on the total weight of the formulation. If vitamin A or vitamin A derivatives or carotenes or derivatives thereof are the antioxidant or the antioxidants, it is advantageous to choose the respective concentrations thereof from the range of from 0.001 to 10% by weight, based on the total weight of the formulation.

In addition to one or more oil phases, cosmetic or dermatological formulations for the purposes of the present application preferably can additionally contain one or more aqueous phases and may be present, e.g., in the form of W/O, O/W, W/O/W or O/W/O emulsions. Emulsions of this type can preferably also be a microemulsion, a pickering emulsion or a sprayable emulsion.

Furthermore, however, the formulations according to the invention can also be present in the form of oil-free preparations, such as, e.g., gels, or as nonaqueous preparations.

Furthermore, the formulations according to the invention can also advantageously contain dihydroxyacetone or nut extracts and other substances that are to maintain the tan, produce it or additionally intensify it. Dihydroxyacetone is then preferably used in a concentration of 0.1-10% by weight, particularly preferably in the range of 0.5-5% by weight.

The lipid phase of the emulsions according to the invention can advantageously be chosen from the following group of substances:
mineral oils, mineral waxes
oils, such as triglycerides of capric or caprylic acid, but preferably castor oil;
fats, waxes and other natural and synthetic fatty substances, preferably esters of fatty acids with alcohols of low carbon number, e.g., with isopropanol, propylene glycol or glycerol, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, from the group of esters of aromatic carboxylic acids and saturated and/or unsaturated, branched and/or unbranched alcohols with a chain length of from 3 to 30 C atoms. Such ester oils can then be advantageously chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters such as, for example, jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and of fatty acid triglycerides, namely the triglycerol esters of saturated and/or unsaturated, branched and/or unbranched alkanecarboxylic acids having a chain length of from 8 to 24, in particular 12 to 18, carbon atoms; The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, for the purposes of the present invention, paraffin oil, squalane and squalene may be used advantageously.

The oil phase can also advantageously have a content of cyclic or linear silicone oils, or be composed entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also to be used advantageously for the purposes of the present invention, for example, hexamethylcyclotrisiloxane, polydimethylsiloxane, poly(methylphenyl-siloxane).

Also particularly advantageous are mixtures of cyclomethicone and isotridecyl isononanoate, of cyclomethicone and 2-ethylhexyl isostearate.

The aqueous phase of the formulations according to the invention may optionally advantageously comprise
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g., ethanol, isopropanol, 1,2-propanediol, glycerol, and, in particular, one or more thickeners which can be chosen advantageously from the group silicon dioxide, aluminum silicates, polysaccharides and derivatives thereof, e.g., hyaluronic acid, xanthan gum, hydroxypropylmethylcellulose, particularly advantageously from the group of polyacrylates, preferably a polyacrylate from the group of so-called Carbopols, e.g., Carbopol grades 980, 981, 1382, 2984, 5984, in each case individually or in combination.

Furthermore, UV filter substances can be added to the preparation according to the invention.

Particularly advantageous UV filter substances which are liquid at room temperature for the purposes of the present invention are homomethyl salicylate (INCI: Homosalate), 2-ethylhexyl 2-cyano-3,3-diphenylacrylate (INCI: octocrylene), 2-ethylhexyl-2-hydroxybenzoate (2-ethylhexyl salicylate, octyl salicylate, INCI: octyl salicylate) and esters of cinnamic acid, preferably 2-ethylhexyl 4-methoxycinnamate (INCI: octyl methoxycinnamate) and isopentyl 4-methoxycinnamate (INCI: isoamyl p-methoxycinnamate).

Preferred inorganic pigments are metal oxides and/or other metal compounds which are insoluble or virtually insoluble in water, in particular the oxides of titanium (TiO₂), zinc (ZnO), iron (e.g., Fe₂O₃), zirconium (ZrO₂), silicon (SiO₂), manganese (e.g., MnO), aluminum (Al₂O₃), cerium (e.g., Ce₂O₃), mixed oxides of the corresponding metals and mixtures of such oxides as well as the sulfate of barium (BaSO₄).

The pigments can also be advantageously used for the purposes of the present invention in the form of commercially available oily or aqueous predispersions. Advantageously, dispersants and/or solubilizers can also be added to these predispersions.

According to the present invention, the pigments can be advantageously surface-treated (coated), whereby, e.g., a hydrophilic, amphiphilic or hydrophobic character is to be formed or retained. This surface treatment can be that the pigments are provided with a thin hydrophilic and/or hydrophobic inorganic and/or organic layer according to methods known per se. The different surface coatings can also contain water for the purposes of the present invention.

Inorganic surface coatings for the purposes of the present invention may comprise aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination and/or in combination with organic coating materials.

Organic surface coatings for the purposes of the present invention may consist of vegetable or animal aluminum stearate, vegetable or animal stearic acid, lauric acid, dimethylpolysiloxane (also: Dimethicone), methylpolysiloxane (Methicone), simethicone (a mixture of dimethylpolysiloxane with an average chain length of from 200 to 350 dimethylsiloxane units and silica gel) or alginic acid. These organic surface coatings may be present individually, in combination and/or in combination with inorganic coating materials.

Zinc oxide particles and predispersions of zinc oxide particles which are suitable according to the invention are obtainable under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |

Suitable titanium dioxide particles and predispersions of titanium dioxide particles are obtainable under the following trade names from the companies listed:

| Trade name | Coating | Manufacturer |
|---|---|---|
| MT-100TV | Aluminum | Tayca Corporation |
| | hydroxide/stearic acid | |
| MT-100Z | Aluminum | Tayca Corporation |
| | hydroxide/stearic acid | |
| Eusolex T-2000 | Alumina/simethicone | Merck KGaA |
| Titanium T805 | Octyltrimethylsilane | Degussa |
| (Uvinul TiO₂) | | |

Advantageous UV A filter substances for the purposes of the present invention are dibenzoylmethane derivatives, in particular 4-(tert-butyl)-4′-methoxydibenzoylmethane (CAS No. 70356-09-1), which is sold by Givaudan under the trade name Parsol® 1789 and by Merck under the trade name Eusolex® 9020.

Further advantageous UV filter substances for the purposes of the present invention are sulfonated, water-soluble UV filters, such as, e.g.,
phenylene-1,4-bis(2-benzimidazyl)-3,3′-5,5′-tetrasulfonic acid and salts thereof, particularly the corresponding sodium, potassium or triethanolammonium salts, in particular the phenylene-1,4-bis(2-benzimidazyl)-3,3′-5,5′-tetrasulfonic acid bis-sodium salt having the INCI name Bisimidazylate (CAS No. 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name phenylbenzimidazole sulfonic acid (CAS No. 27503-81-7), which is available, for example, under the trade name Eusolex 232 from Merck or under the trade name Neo Heliopan Hydro from Haarmann & Reimer;
1,4-di(2-oxo-10-sulfo-3-bornylidenemethyl)-benzene (also: 3,3′-(1,4-phenylenedimethylene)-bis-(7,7-dimethyl-2-oxo-bicyclo-[2.2.1]hept-1-ylmethane sulfonic acid) and salts thereof (in particular the corresponding 10-sulfato compounds, in particular the corresponding sodium, potassium or triethanolammonium salt), which is also known as benzene-1,4-di(2-oxo-3-bornylidenemethyl-10-sulfonic acid). Benzene-1,4-di(2-oxo-3-bornylidenemethyl-10-sulfonic acid) has the INCI name terephthalidene dicamphor sulfonic acid (CAS No.: 90457-82-2) and is available, for example, under the trade name Mexoryl SX from Chimex;
sulfonic acid derivatives of 3-benzylidene camphor, such as, e.g., 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention include furthermore so-called broadband filters, i.e., filter substances which absorb both UVA and UVB radiation.

Advantageous broadband filters or UVB filter substances include, for example, triazine derivatives, such as e.g.
2,4-bis-{[4-(2-ethylhexyloxy)-2-hydroxy]-phenyl}-6-(4-methoxyphenyl)-1,3,5-triazine (INCI: aniso triazine), which is available under the trade name Tinosorb® S from CIBA-Chemikalien GmbH;
Diethylhexylbutylamidotriazone (INCI: diethylhexylbutamidotriazone), which is available under the trade name UVASORB HEB from Sigma 3V;
tris(2-ethylhexyl) 4,4′,4″-(1,3,5-triazine-2,4,6-triyltriimino)-tris-benzoate, also: 2,4,6-tris-[anilino-(p-carbo-2′-ethyl-1′-hexyloxy)]-1,3,5-triazine (INCI: ethylhexyl triazone), which is sold by BASF Aktiengesellschaft under the trade name UVINUL® T 150.

Another advantageous broadband filter for the purposes of the present invention is 2,2′-methylene-bis-(6-(2H-benzotriazol-2-yl)-4-(1,1,3,3-tetramethylbutyl)phenol) which is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]-disiloxanyl]propyl]-phenol (CAS No.: 155633-54-8) with the INCI name drometrizole trisiloxane, which is available under the trade name Mexoryl® XL from Chimex.

The further UV filter substances may be oil-soluble or water-soluble. Advantageous oil-soluble UVB and/or broadband filter substances for the purposes of the present invention include, e.g.:
3-benzylidene camphor derivatives, preferably 3-(4-methylbenzylidene) camphor, 3-benzylidene camphor;
4-aminobenzoic acid derivatives, preferably (2-ethylhexyl) 4-(dimethylamino)benzoate, amyl 4-(dimethylamino)benzoate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
UV filters bound to polymers;
3-(4-(2,2-bisethoxycarbonylvinyl)-phenoxy)propenyl)-methoxysiloxane/dimethylsiloxane copolymer, which is available, e.g., under the trade name Parsol® SLX from Hoffmann La Roche.

Advantageous water-soluble filter substances include, e.g., sulfonic acid derivatives of 3-benzylidene camphor, such as, e.g., 4-(2-oxo-3-bornylidenemethyl)benzenesulfonic acid, 2-methyl-5-(2-oxo-3-bornylidenemethyl)sulfonic acid and salts thereof.

A further light-protection filter substance which may advantageously be used according to the invention is ethylhexyl-2-cyano-3,3-diphenylacrylate (octocrylene), which is available from BASF under the name Uvinul® N 539.

Particularly advantageous preparations for the purposes of the present invention which are characterized by a high or very high UVA and/or UVB protection furthermore preferably comprise, in addition to the filter substance(s) according to the invention, further UVA and/or broadband filters, in particular dibenzoylmethane derivatives [for example, 4-(tert-butyl)-4′-methoxydibenzoylmethane], phenylene-1,4-bis-(2-benzimidazyl)-3,3′-5,5′-tetrasulfonic acid and salts thereof, 1,4-di(2-oxo-10-sulfo-3-bornylidenemethyl)-benzene and/or salts thereof and/or 2,4-bis-{[4-(2-ethylhexyloxy)-2-hydroxy]-phenyl}-6-(4-methoxyphenyl)-1,3,5-triazine, in each case individually or in any desired combinations with one another.

Furthermore, particularly advantageous according to the invention are benzoxazole derivatives, such as in particular 2,4-bis[5-1(dimethylpropyl)benzoxazol-2-yl-(4-phenyl)imino]-6-(2-ethylhexyl)imino-1,3,5-triazine with the CAS No. 288254-16-0, which is available, for example, under the trade name Uvasorb® K2A, and hydroxybenzophenones such as in particular the hexyl 2-(4′-diethylamino-2′-hydroxybenzoyl)-benzoate or aminobenzophenone, which is available under Uvinul A Plus.

The above list of UV filters which can be employed for the purposes of the present invention is of course not intended to be limiting.

The preparations according to the invention may advantageously comprise the substances which absorb UV radiation in the UVA and/or UVB range in a total amount of, e.g., from 0.1% by weight to 30% by weight, preferably from 0.5% to 20% by weight, in particular from 1.0% to 15.0% by weight, in each case based on the total weight of the preparations, in order to provide cosmetic preparations which protect the hair or the skin from the entire range of ultraviolet radiation. They can also be used as sunscreen for the hair.

It is also advantageous, although not obligatory, for the formulations according to the invention to be used in combination with UV radiation—whether with artificially produced or natural UV rays—e.g., in order to increase the natural tan or in order to achieve a particularly long-lasting tan.

The cosmetic and dermatological preparations according to the invention are applied for use to the skin and/or the hair in sufficient quantity in the customary manner for cosmetics.

According to the extensive explanations, the use of the composition according to the invention, in particular a cosmetic and/or dermatological preparation, is preferred
As an aqueous system and/or surfactant preparation for cleansing and care of the skin and/or hair,
As a multiple emulsion, microemulsion, pickering emulsion or sprayable emulsion,
As a pre-sun, sunscreen or an after-sun formulation,
For topical application on the skin and/or hair,
For tanning the skin,
For the care of the skin,
To protect the skin and/or hair from harmful UV rays,
To increase melanin synthesis in the skin,
To prolong the tanning of the skin,
To protect the skin from oxidative stress,
To protect the skin from chronological and light-related aging of the skin,
To intensify the color of the hair,
To prevent graying of the hair and/or to protect the hair from bleaching by the sun,
As a shower gel, shampoo, conditioner, hair treatment, hair rinse, hair tonic, hairspray, make up, skin protection, face, cleansing, sunscreen, nourishing, day or night cream, gel or lotion or cleansing preparation.

The compounds according to the invention can also be a constituent of a polymer matrix, a skin patch and/or a wound dressing, a plaster, a wipe or a pad, a spray or be applied in textiles, such as bandages or bath textiles.

The incorporation of the compounds into polymer matrices, such as polyurethane matrices, is thus easily possible. Similar to the known release of active ingredients, the compounds can be released from the matrix onto the skin or the hair and there render possible their advantageous properties. In a plaster application or applied on textiles, bandages or the like, the compounds can penetrate into the skin and produce the desired protective, caring or tanning effect.

An application as a spray is preferred, since here the compounds merely have to be mixed with suitable aerosols or gases.

All the amounts, proportions and percentages given in the following examples are, unless specified otherwise, based on the weight and the total amount or on the total weight of the preparations.

PIT Emulsions

| | Example | | | | |
|---|---|---|---|---|---|
| | 1 | 2 | 3 | 4 | 5 |
| Glycerin monostearate self-emulsifying | 0.50 | | 3.00 | 2.00 | 4.00 |
| Polyoxyethylene (12) cetylstearyl ether | | 5.00 | | 1.00 | 1.50 |
| Polyoxyethylene (20) cetylstearyl ether | | | | 2.00 | |
| Polyoxyethylene (30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxy cinnamate | | | | 5.00 | 8.00 |
| 2,4-Bis-(4-(2-ethyl-hexyloxy)-2-hydroxyl)- | | 1.50 | | 2.00 | 2.50 |
| phenyl)-6-(4-methoxyphenyl)-(1,3,5)- | | | | | |
| triazine | | | | | |
| Butylmethoxy-dibenzoylmethane | | | 2.00 | | |
| Diethylhexyl Butamidotriazone | 1.00 | 2.00 | | 2.00 | |
| Ethylhexyl Triazone | 4.00 | | 3.00 | 4.00 | |
| 4-Methylbenzylidene camphor | | 4.00 | | | 2.00 |
| Octocrylene | | 4.00 | | | 2.50 |
| Phenylene-1,4-bis-(monosodium,2- | | | 0.50 | | 1.50 |
| benzimidazyl-5,7-disulfonic acid | | | | | |
| Phenylbenzimidazole sulfonic acid | 0.50 | | | 3.00 | |
| C12-15 Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butyleneglycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethyl polysiloxane | 2.00 | | | 0.50 | 0.50 |
| Shea butter | | 2.00 | | | 0.50 |
| PVP Hexadecene copolymer | 0.50 | | | 0.50 | 1.00 |
| Glycerin | 3.00 | 7.50 | 5.00 | 7.50 | 2.50 |
| Tocopherol acetate | 0.50 | | 0.25 | | 1.00 |
| (4E,8E,12E,16E)-3,7,11,15-tetrahydroxy-18- | 0.05 | | 1.00 | 0.20 | 0.10 |
| (hydroxymethyl)-2,4,6,10,14,16,20- | | | | | |
| heptamethyldocosa-4,8,12,16-tetraenoic acid | | | | | |
| alpha-Glucosylrutin | 0.10 | | 0.20 | | |
| (2R,3S,4R)-2,3,4-trimethyl-3-[(3E,7E,11E)- | | 0.30 | | | 0.20 |
| 3,8,12,16-tetramethylheptadeca-3,7,11,15- | | | | | |
| tetraen-lyl]cyclohexanone | | | | | |
| Preservatives | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

O/W Cream

| | Examples | | | | |
|---|---|---|---|---|---|
| | 6 | 7 | 8 | 9 | 10 |
| Glyceryl stearate citrate | | | 2.00 | | 2.00 |
| Glyceryl stearate self-emulsifying | 4.00 | 3.00 | | | |
| PEG-40 Stearate | 1.00 | | | | |
| Polyglyceryl 3-methylglucose distearate | | | | 3.00 | |
| Sorbitan stearate | | | | | 2.00 |
| Stearic acid | | 1.00 | | | |
| Polyoxyethylene (20) cetylstearyl ether | | | | | |
| Stearyl alcohol | | | 5.00 | | |
| Cetyl alcohol | 3.00 | 2.00 | | 3.00 | |
| Cetylstearyl alcohol | | | | | 2.00 |
| C12-15 Alkyl benzoate | | | | | |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidene camphor | | | 1.00 | | |
| Butylmethoxy dibenzoylmethane | | | 0.50 | | |
| (2R,3S,4R)-2,3,4-trimethyl-3-[(3E,7E,11E)- | 0.25 | 0.05 | | | 0.05 |
| 3,8,12,16-tetramethylheptadeca-3,7,11,15- | | | | | |
| tetraen-lyl]cyclohexanone | | | | | |
| Tocopherol | 0.1 | | | | 0.20 |
| (4E,8E,12E,16E)-3,7,11,15-tetrahydroxy-18- | 0.05 | | 0.1 | 0.15 | |
| (hydroxymethyl)-2,4,6,10,14,16,20- | | | | | |
| heptamethyldocosa-4,8,12,16-tetraenoic acid | | | | | |
| Biotin | | | 0.05 | | |
| Ethylenediamine tetraacetic acid trisodium | 0.1 | | 0.10 | 0.1 | |
| Preservative | q.s. | q.s. | q.s. | q.s. | q.s. |
| Xanthan gum | | | | | |
| Polyacrylic acid | 3.00 | 0.1 | | 0.1 | 0.1 |
| Aqueous sodium hydroxide 45% | q.s. | q.s. | q.s. | q.s. | q.s. |
| Glycerin | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |

O/W Cream

| | Examples | | | | |
|---|---|---|---|---|---|
| | 11 | 12 | 13 | 14 | 15 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 Alkyl benzoate | | 2.00 | 3.00 | | |
| Caprylic/capric triglyceride | 2.00 | | | | |
| Octyldodecanol | 2.00 | 2.00 | | 4.00 | 6.00 |
| N-Acetyl tyrosine | 0.5 | | | 0.1 | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| Phytoene | | 0.10 | | 0.20 | |
| 4-Methylbenzylidene camphor | 1.00 | | | | 1.00 |
| Butylmethoxy dibenzoylmethane | 0.50 | | | | 0.50 |
| (4E,8E,12E,16E)-3,7,11,15-tetrahydroxy-18- | 0.08 | 0.50 | 0.25 | 0.3 | 0.40 |
| (hydroxymethyl)-2,4,6,10,14,16,20- | | | | | |
| heptamethyldocosa-4,8,12,16-tetraenoic acid | | | | | |
| 2,4-Bis-(4-(2-ethyl-hexyloxy)-2-hydroxyl)- | | 1.0 | 3.0 | | 0.5 |
| phenyl)-6-(4-methoxyphenyl)-(1,3,5)-triazine | | | | | |
| Dihydroxyacetone | | 0.5 | | | 0.5 |
| Tocopherol | | | | | 0.05 |
| Ethylenediamine tetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Aqueous sodium hydroxide 45% | q.s. | q.s. | q.s. | q.s. | q.s. |
| Glycerin | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

W/O Emulsions

| | 16 | 17 | 18 | 19 | 20 |
|---|---|---|---|---|---|
| Cetyl dimethicone copolyol | | 2.50 | | 4.00 | |
| Polyglyceryl 2-dipolyhydroxystearate | 5.00 | | | | 4.50 |
| PEG-30 Dipolyhydroxystearate | | | 5.00 | | |
| 2-Ethylhexyl Methoxycinnamate | | 8.00 | | 5.00 | 4.00 |
| 2,4-Bis-(4-(2-ethyl-hexyloxy)-2-hydroxyl)- | 2.00 | 2.50 | | 2.00 | 2.50 |
| phenyl)-6-(4-methoxyphenyl)-(1,3,5)-triazine | | | | | |
| Butylmethoxy dibenzoylmethane | | | 2.00 | 1.00 | |
| Diethylhexyl Butamidotriazone | 3.00 | 1.00 | | | 3.00 |
| Ethylhexyl Triazone | | | 3.00 | 4.00 | |
| 4-Methylbenzylidene camphor | | 2.00 | | 4.00 | 2.00 |
| Octocrylene | 7.00 | 2.50 | 4.00 | | 2.50 |
| N-Acetyl tyrosine | | 0.20 | 0.30 | | |
| Diethylhexyl Butamidotriazone | 1.00 | | | 2.00 | |
| Phenylene-1,4-bis-(monosodium,2- | 1.00 | 2.00 | 0.50 | | |
| benzimidazyl-5,7-disulfonic acid) | | | | | |
| Phenylbenzimidazole sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| Dihydroxyacetone | | 0.7 | | 0.5 | 0.5 |
| C12-15 Alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethyl polysiloxane | 2.00 | 25.00 | | | 2.00 |
| Shea butter | | | 3.00 | | |
| PVP Hexadecene copolymer | 0.50 | | | 0.50 | 1.00 |
| Octoxyglycerin | | 0.30 | 1.00 | | 0.50 |
| Glycerin | 3.00 | 7.50 | | 7.50 | 2.50 |
| Glycin soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| (4E,8E,12E,16E)-3,7,11,15-tetrahydroxy-18- | 0.15 | 0.08 | 0.5 | 1.00 | 0.80 |
| (hydroxymethyl)-2,4,6,10,14,16,20- | | | | | |
| heptamethyldocosa-4,8,12,16-tetraenoic acid | | | | | |
| Phytoen | 0.20 | | 0.01 | | 0.05 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

W/O Emulsions

Example

| | 21 | 22 |
|---|---|---|
| Polyglyceryl 2-dipolyhydroxystearate | 4.00 | 5.00 |
| PEG-30 Dipolyhydroxystearate | | |
| Lanolin alcohol | 0.50 | 1.50 |
| Isohexadecane | 1.00 | 2.00 |
| Myristyl myristate | 0.50 | 1.50 |
| Petrolatum | 1.00 | 2.00 |
| Butylmethoxy dibenzoylmethane | 0.50 | 1.50 |
| 4-Methylbenzylidene camphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerin | | 3.00 |
| Glycerin | 5.00 | |
| (4E,8E,12E,16E)-3,7,11,15-tetrahydroxy- | 0.50 | 0.50 |
| 18-(hydroxymethyl)-2,4,6,10,14,16,20- | | |
| heptamethyldocosa-4,8,12,16-tetraenoic | | |
| acid | | |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

Hydrodispersions

| | Example | | | | |
|---|---|---|---|---|---|
| | 23 | 24 | 25 | 26 | 27 |
| Polyoxyethylene (20) cetylstearyl ether | 1.00 | | | 0.5 | |
| Cetyl alcohol | | | 1.00 | | |
| Sodium polyacrylate | | 0.20 | | 0.30 | |
| Acrylate/C10-30 Alkyl Acrylate | 0.50 | | 0.40 | 0.10 | 0.10 |
| Crosspolymer | | | | | |
| Xanthan gum | | 0.30 | 0.15 | | 0.50 |
| 2-Ethylhexyl Methoxycinnamate | | | | 5.00 | 8.00 |
| 2,4-Bis-(4-(2-ethyl-hexyloxy)-2-hydroxyl)- | | 1.50 | | 2.00 | 2.50 |
| phenyl)-6-(4-methoxyphenyl)-(1,3,5) | | | | | |
| triazine | | | | | |
| Butylmethoxy dibenzoylmethane | 1.00 | | 2.00 | | |
| Diethylhexyl Butamidotriazone | | 2.00 | | 2.00 | 1.00 |
| Ethylhexyl Triazone | 4.00 | | 3.00 | 4.00 | |
| 4-Methylbenzylidene camphor | 4.00 | 4.00 | | | 2.00 |
| Octocrylene | | 4.00 | 4.00 | | 2.50 |
| Phenylene-1,4-bis-(monosodium,2- | 1.00 | | 0.50 | | 2.00 |
| benzimidazyl-5,7-disulfonic acid | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 Alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethyl polysiloxane | 2.00 | | | 0.50 | 2.00 |
| Shea butter | | 2.00 | | | |
| PVP Hexadecene copolymer | 0.50 | | | 0.50 | 1.00 |
| Octoxyglycerin | | | 1.00 | | 0.50 |
| Glycerin | 3.00 | 7.50 | | 7.50 | 2.50 |
| Glycin soya | | | 1.50 | | |
| Tocopherol acetate | 0.50 | | 0.25 | | 1.00 |
| (4E,8E,12E,16E)-3,7,11,15-tetrahydroxy-18- | 0.15 | 0.50 | 0.05 | 1.00 | 0.40 |
| (hydroxymethyl)-2,4,6,10,14,16,20- | | | | | |
| heptamethyldocosa-4,8,12,16-tetraenoic acid | | | | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

### EXAMPLE 28

### Gel Cream

| | |
|---|---|
| Acrylate/C10-30 Alkyl acrylate | 0.40 |
| Crosspolymer | |
| Polyacrylic acid | 0.20 |
| Xanthan gum | 0.10 |
| Cetearyl alcohol | 3.00 |
| C12-15 Alkyl benzoate | 4.00 |
| Caprylic/capric triglyceride | 3.00 |
| Cyclic dimethylpolysiloxane | 5.00 |
| Dimeticone polydimethylsiloxane | 1.00 |
| (4E,8E,12E,16E)-3,7,11,15-tetrahydroxy- | 0.1 |
| 18-(hydroxymethyl)-2,4,6,10,14,16,20- | |
| heptamethyldocosa-4,8,12,16-tetraenoic | |
| acid | |
| Glycerin | 3.00 |
| Sodium hydroxide | q.s. |
| Preservative | q.s. |
| Perfume | q.s. |
| Water | ad 100.0 |

pH adjusted to 6.0

### EXAMPLE 29

### W/O Cream

| | |
|---|---|
| Polyglyceryl 3-diisostearates | 3.50 |
| Glycerin | 3.00 |
| Polyglyceryl 2-dipolyhydroxystearates | 3.50 |
| (4E,8E,12E,16E)-3,7,11,15-tetrahydroxy- | 0.25 |
| 18-(hydroxymethyl)-2,4,6,10,14,16,20- | |
| heptamethyldocosa-4,8,12,16-tetraenoic | |
| acid | |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

### EXAMPLE 30

### W/O/W Cream

| | |
|---|---|
| Glyceryl stearate | 3.00 |
| PEG-100 Stearate | 0.75 |
| Behenyl alcohol | 2.00 |
| Caprylic/capric triglyceride | 8.0 |
| Octyldodecanol | 5.00 |
| C12-15 Alkyl benzoate | 3.00 |
| (4E,8E,12E,16E)-3,7,11,15-tetrahydroxy-18- | 0.5 |
| (hydroxymethyl)-2,4,6,10,14,16,20- | |
| heptamethyldocosa-4,8,12,16-tetraenoic acid | |
| Magnesium sulfate (MgSO4) | 0.80 |
| Ethylene diamine tetraacetic acid | 0.10 |
| Preservative | q.s. |
| Perfume | q.s. |
| Water | ad 100.0 |

pH adjusted to 6.0

### EXAMPLE 31

### Spray Formulation

| | |
|---|---|
| Ethanol | 28.00 |
| (4E,8E,12E,16E)-3,7,11,15-tetrahydroxy-18- | 0.10 |
| (hydroxymethyl)-2,4,6,10,14,16,20- | |
| heptamethyldocosa-4,8,12,16-tetraenoic acid | |
| Preservatives, dyes, perfume | q.s. |
| Propane/butane 25/75 | ad 100 |

### EXAMPLE 32

### Shower Bath

| | |
|---|---|
| Sodium laureth sulfate | 33.00 |
| Potassium cocoyl hydrogenated collagen | 11.00 |
| (30%) | |
| Cocoamphodiacetate (30%) | 5.00 |
| PEG-7 Glyceryl Cocoate | 2.00 |
| Cocamide MEA | 1.00 |
| Sodium chloride | 0.50 |
| (4E,8E,12E,16E)-3,7,11,15-tetrahydroxy-18- | 0.05 |
| (hydroxymethyl)-2,4,6,10,14,16,20- | |
| heptamethyldocosa-4,8,12,16-tetraenoic acid | |
| Citric acid | 0.02 |
| Preservatives, dyes, perfume | q.s. |
| Water | ad 100 |

### EXAMPLE 33

### Hair Treatment

| | |
|---|---|
| Hydroxypropylmethyl cellulose | 0.50 |
| Cetrimonium bromide | 1.00 |
| Glycerin | 3.00 |
| Cetearyl alcohol | 2.50 |
| Benzophenone-4 | 0.4 |
| Glyceryl stearate | 2.00 |
| (4E,8E,12E,16E)-3,7,11,15-tetrahydroxy-18- | 0.1 |
| (hydroxymethyl)-2,4,6,10,14,16,20- | |
| heptamethyldocosa-4,8,12,16-tetraenoic acid | |
| Preservatives, perfume, pH adjustment | q.s. |
| Water | ad 100 |
| The pH is adjusted to 3.5. | |

### EXAMPLE 34

### Hair Rinse

| | |
|---|---|
| Behentrimonium chloride | 1.00 |
| Glycerin | 3.00 |
| Benzophenone-4 | 0.25 |
| Hydroxyethyl cellulose | 0.20 |
| Cetearyl alcohol | 3.00 |
| (4E,8E,12E,16E)-3,7,11,15-tetrahydroxy-18- | 0.2 |
| (hydroxymethyl)-2,4,6,10,14,16,20- | |
| heptamethyldocosa-4,8,12,16-tetraenoic acid | |
| Phytoene | 0.80 |
| Preservatives, perfume, pH adjustment | q.s. |
| Water | ad 100 |
| The pH is adjusted to 3.0. | |

Conditioner Shampoo with Pearlescence

| | Example | | |
|---|---|---|---|
| | 35 | 36 | 37 |
| Polyquaternium-10 | 0.5 | 0.5 | 0.5 |
| Sodium laureth sulfate | 9.0 | 9.0 | 9.0 |
| Benzophenone-3 | | 0.5 | |
| Benzophenone-4 | | | 0.4 |
| Cocoamidopropyl betaine | 2.5 | 2.5 | 2.5 |
| Pearlescent | 2.0 | 2.0 | 2.0 |
| (4E,8E,12E,16E)-3,7,11,15- | 0.06 | 0.15 | 0.01 |
| tetrahydroxy-18-(hydroxymethyl)- | | | |
| 2,4,6,10,14,16,20- | | | |
| heptamethyldocosa- | | | |
| 4,8,12,16-tetraenoic | | | |
| acid | | | |
| Disodium EDTA | 0.1 | 0.2 | 0.15 |
| Preservative, perfume, thickener, pH | q.s. | q.s. | q.s. |
| adjustment and solubilizer | | | |
| Water, VES (completely | ad 100.0 | ad 100.0 | ad 100.0 |
| demineralized) | | | |
| The pH is adjusted to 6. | | | |

Clear Conditioner Shampoo

| | Example | | |
|---|---|---|---|
| | 38 | 39 | 40 |
| Polyquaternium-10 | 0.5 | 0.5 | 0.5 |
| Benzophenone-4 | | 0.4 | |
| 2-Ethylhexyl Methoxycinnamate | | | 0.2 |
| Sodium laureth sulfate | 9.0 | 9.0 | 9.0 |
| Cocoamidopropyl betaine | 2.5 | 2.5 | 2.5 |
| (4E,8E,12E,16E)-3,7,11,15- | 0.02 | 0.05 | 0.05 |
| tetrahydroxy-18-(hydroxymethyl)- | | | |
| 2,4,6,10,14,16,20- | | | |
| heptamethyldocosa- | | | |
| 4,8,12,16-tetraenoic | | | |
| acid | | | |
| Iminodisuccinic acid, Na-salt | 0.2 | 0.3 | 0.8 |
| Preservative, perfume, thickener, pH | q.s. | q.s. | q.s. |
| adjustment and solubilizer | | | |
| Water, VES (completely | ad 100.0 | ad 100.0 | ad 100.0 |
| demineralized) | | | |
| The pH is adjusted to 6. | | | |

Clear Light Shampoo with Volume Effect

| | Example | | |
|---|---|---|---|
| | 41 | 42 | 43 |
| Sodium laureth sulfate | 10.0 | 10.0 | 10.0 |
| Cocoamidopropyl betaine | 2.5 | 2.5 | 2.5 |
| (4E,8E,12E,16E)-3,7,11,15- | 0.5 | 0.6 | 0.3 |
| tetrahydroxy-18-(hydroxymethyl)- | | | |
| 2,4,6,10,14,16,20- | | | |
| heptamethyldocosa- | | | |
| 4,8,12,16-tetraenoic | | | |
| acid | | | |
| Disodium EDTA | 0.2 | 0.15 | 0.7 |
| Preservative, perfume, thickener, pH | q.s. | q.s. | q.s. |
| adjustment and solubilizer | | | |
| Water, VES (completely | ad 100.0 | ad 100.0 | ad 100.0 |
| demineralized) | | | |
| The pH is adjusted to 5.5. | | | |

1.-24. (canceled)

25. A composition which is suitable for use on skin or hair, wherein the composition comprises one or more of the following compounds:
(I) (2R,3S,4R)-2,3,4-trimethyl-3-[(3E,7E,11E)-3,8,12,16-tetramethylheptadeca-3,7,11,15-tetraen-1-yl]cylcohexanone of formula 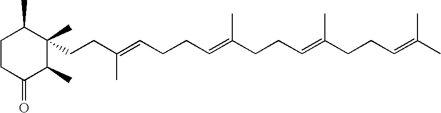
(II) (2E,6E)-10-hydroxy-2,6,10-trimethyldodeca-2,6,1,1-trien-1-yl acetate of formula 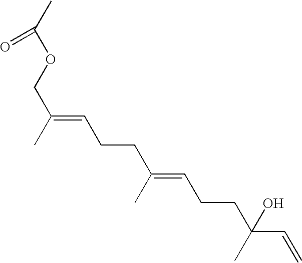
(III) (4E,8E,12E,16E)-3,7,11,15-tetrahydroxy-18-(hydroxymethyl)-2,4,6,10,14,16,20-heptamethyldocosa-4,8,12,16-tetraenoic acid of formula 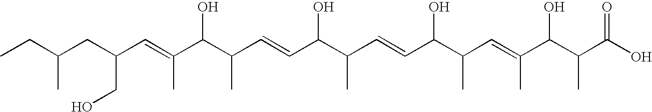
(IV) (3E)-7-hydroxy-1-[(1Z)-3-hydroxy-2-methylprop-1-en-1-yl]-3,7-dimethylnona-3,8-dien-1-yl acetate of formula 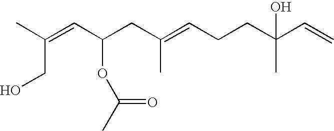
(V) (2E,6Z)-10-hydroxy-2,6,10-trimethyldodeca-2,6,11-trienoic acid of formula 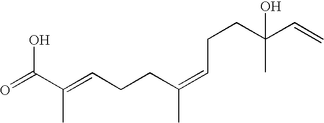
(VI) 6-methyl-8-(2,5,5,8a-tetramethyl-1,4,4a,5,6,7,8,8a-octahydronaphthalen-1-yl)oct-5-en-2-one of formula 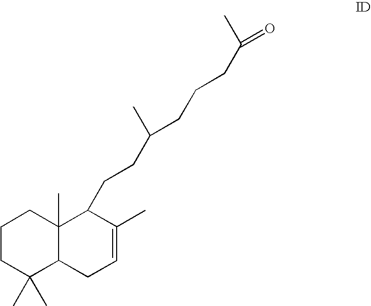
(VII) (2E,6E,10E)-13-[(1R,2R,8aS)-2-hydroxy-2,5,5,8a-tetramethyl-6-oxodeca-hydronaphthalen-1-yl]-2,6,10-trimethyltrideca-2,6,10-trienoic acid of formula and 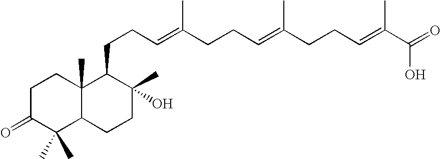
(VIII) (4aS,5R,6R)-6-hydroxy-5-[(3E,7E,11E)-13-hydroxy-4,8,12-trimethyltrideca-3,7,11-trien-1-yl]-1,1,4a,6-tetramethyloctahydronaphthalen-2(1H)-one of formula 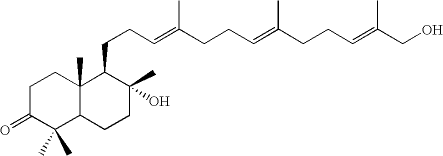
and at least one other component or element.

26. The composition of claim 25, wherein the composition comprises a compound of formula (III).

27. The composition of matter of claim 25, wherein the composition comprises a cosmetic preparation, a dermatological preparation, a polymer matrix, a skin covering, a wound dressing, a plaster, a wipe, a pad, a spray, a stick or a textile.

28. The composition of matter of claim 27, wherein the composition comprises a cosmetic preparation or a dermatological preparation.

29. The composition of claim 25, wherein the composition comprises one or more of the compounds of formulae (I) to (VIII) in a total amount of from 0.0001% to 30% by weight, based on a total weight of the composition.

30. The composition of claim 29, wherein the composition comprises one or more of the compounds of formulae (I) to (VIII) in a total amount of from 0.001% to 10% by weight.

31. The composition of claim 28, wherein the composition comprises one or more of the compounds of formulae (I) to (VIII) in a total amount of from 0.02% to 2% by weight, based on a total weight of the composition.

32. The composition of claim 25, wherein the composition comprises one or more of the compounds of formulae (I) to (VIII) in encapsulated form.

33. The composition of claim 32, wherein one or more of the compounds of formulae (I) to (VIII) are encapsulated in a material comprising one of more of a collagen matrix, a cyclic oligosaccharide, alpha-, beta-, HP-beta-, random-Me-beta, gamma-cylcodextrin, cellulose, gelatin, a wax matrix and liposomes.

34. The composition of claim 25, wherein the composition further comprises at least one of a UVA filter, a UVB filter and an inorganic pigment.

35. The composition of claim 34, wherein the composition comprises an inorganic micropigment.

36. The composition of claim 25, wherein the composition further comprises one or more antioxidants in a total amount of from 0.001% to 30% by weight, based on a total weight of the composition.

37. The composition of claim 36, wherein the composition comprises from 0.05% to 20% by weight of the one or more antioxidants.

38. The composition of claim 36, wherein the composition comprises from 0.1% to 10% by weight of the one or more antioxidants.

39. The composition of claim 25, wherein the composition further comprises glycerol in an amount of from 0.05% to 30% by weight, based on a total weight of the composition.

40. The composition of claim 39, wherein the composition comprises glycerol in an amount of from 1% to 10% by weight.

41. The composition of claim 25, wherein the composition further comprises one or more of phytoene, phytofluene, ζ-carotene, neurosporine, lycopene, α-carotene, squalene, variabilin, phytanic acid and phytol.

42. The composition of claim 25, wherein the composition comprises one or more components selected from active ingredients which have a positive effect on the condition of the skin, promoting agents for restructuring connective tissue, active ingredients for assisting skin functions in cases of dry skin, active ingredients for at least one of alleviating and positively influencing irritated skin conditions, inhibitors of prostaglandin metabolism, modulators of pigmentation, and active ingredients which bring about an enhanced or more rapid tanning of skin.

43. The composition of claim 42, wherein the composition comprises one or more components selected from bioquinones, creatine, creatinine, carnitine, biotin, isoflavones, cardiolipin, lipoic acid, antifreezing proteins, arctiin, hop extracts, hop-malt extracts, isoflavonoids, vitamin C, propionic acid, green tea extracts, eucalyptus oil, urea, mineral salts, sea minerals, osmolytes, sericosides, extracts of licorice, licochalcones, silymarin, silyphos, dexpanthenol, inhibitors of cyclooxygenase metabolism, inhibitors of leukotriene metabolism, FLAP, tyrosine sulfate, dioic acid, liponamide, kojic acid, hydroquinone, arbutin, fruit acids, bearberry (Uvae ursi), ursolic acid, aminoguanidine, pyridoxamine, Advanced Glycation Endproducts (AGE), lipofuscins, nucleic acid oligonucleotides, purines, pyrimidines, dihydroxyacetone, erythrulose and NO-releasing substances.

44. The composition of claim 25, wherein the composition further comprises at least one component selected from preservatives, bactericides, perfumes, substances for preventing foaming, dyes, fillers, pigments having a coloring effect, thickeners, humectant and/or moisturizing substances, fats, oils, waxes, alcohols, polyols, polymers, foam stabilizers, electrolytes, organic solvents, silicone derivatives, moisturizers, vitamins, proteins, light protection agents, stabilizers, insect repellents, water, salts, substances having anti-microbial, proteolytic or keratolytic activity, and medicaments.

45. The composition of claim 25, wherein the composition comprises an emulsion.

46. The composition of claim 45, wherein the composition comprises at least one of a multiple emulsion, a microemulsion, a Pickering emulsion and a sprayable emulsion.

47. The composition of claim 25, wherein the composition comprises at least one of an aqueous system and a surfactant preparation for at least one of cleansing and caring of skin and/or hair.

48. The composition of claim 25, wherein the composition is in a form which is suitable for topical application to at least one of skin and hair.

49. The composition of claim 48, wherein the composition is in a form of a pre-sun formulation, a sunscreen formulation and an after-sun formulation.

50. The composition of claim 48, wherein the composition is in a form of a shower gel, a shampoo, a conditioner, a hair treatment, a hair rinse, a hair tonic, a hairspray, a make up product, a skin protection product, a face cream, a cleansing cream, a nourishing cream, a day or night cream, a gel, a lotion or a cleansing preparation.

51. The composition of claim 25, wherein the composition is capable of at least one of increasing skin tanning and melanin synthesis in at least one of skin and hair.

52. A method of tanning of skin, wherein the method comprises applying the composition of claim 25 to skin.

53. A method of prolonging tanning of skin, wherein the method comprises applying the composition of claim 25 to skin.

54. A method of increasing synthesis of melanin in skin or hair, wherein the method comprises applying the composition of claim 25 to skin or hair.

55. A method of protecting skin or hair from harmful UV rays, wherein the method comprises applying the composition of claim 25 to the skin or hair to be protected.

56. A method of reducing uneven pigmentation of skin, wherein the method comprises applying the composition of claim 25 to skin.

57. A method of protecting skin against oxidative stress, wherein the method comprises applying the composition of claim 25 to skin.

58. A method of protecting skin against at least one of chronological and photo-induced skin aging and acute damage due to UV radiation, wherein the method comprises applying the composition of claim 25 to skin.

59. A method of intensifying hair color, wherein the method comprises applying the composition of claim 25 to hair.

60. A method at least one of preventing graying of hair and protecting hair against bleaching caused by sunlight, wherein the method comprises applying the composition of claim 25 to hair.
